(19) United States
(12) Patent Application Publication
(10) Pub. No.: US 2026/0263709 A1
(43) Pub. Date: Sep. 10, 2026

(54) MEDICATION DISPENSER

(71) Applicant: Van Asher, Oak Park, IL (US)

(72) Inventor: Van Asher, Oak Park, IL (US)

(21) Appl. No.: 19/289,672

(22) Filed: Aug. 4, 2025

Related U.S. Application Data
(60) Provisional application No. 63/769,463, filed on Mar. 10, 2025.

(51) Int. Cl. A61M 15/00; A61M 15/08

(52) U.S. Cl. CPC A61M 15/0003; A61M 15/0035; A61M 15/08

(57) ABSTRACT

A device for dispensing medicine includes a dispensing nozzle. A base unit includes a base wall and side wall. The dispensing nozzle extends from the base wall, the side wall extends from the base wall forming a pool. The side wall includes recesses on opposing sides thereof. A dispensing apparatus includes an actuator and medication dispensing device. The actuator includes arms extending outwardly therefrom. The medication retaining device retains two doses of medication. In a first position, the arms extend between the recesses. In the second position, the arms extend parallel to the recesses. In the first position, the actuator may be depressed until the arms rest against a base of the recesses dispensing a first dose of medication. In the second position, the actuator may be depressed so the arms rest against the base wall, dispensing a second dose of medication.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of dispensing medication and, more specifically, to a medication dispenser able to dispense at least two separate doses of medication.

### RELATED ART

Presently, many devices are provided for dispensing medication to a user. A number of these devices are able to discharge a dose of medication into the nostril of a user. These devices use many different mechanisms for delivering medication to the user.

For example, U.S. Pat. No. 11,039,982 to Spurr describes a medication delivery device involving an ampule having a receptacle base secured to a tab-shaped cap. The exterior surface between the base and cap is scored to permit a user to twist the cap in a first direction thereby severing the cap from the base and permitting medication secured within the base to be accessed. Adjacent the cap on an interior of the ampule is a unidirectional valve which moderates the ejection of fluid into at least two (2) doses. An exterior portion of the base has a surface suitable for any desired indicia.

US Patent Publication No. 2023/0310752 to Hubert describes a drug delivery device including a mechanical stop configured to provide a pause between the delivery of the first and second doses. During the pause, a user of the drug delivery device can move the drug delivery device from one nostril, into which the first dose was sprayed, to another nostril, into which the second dose can be sprayed. The mechanical stop is configured to hold the drug delivery device in a static delivery state.

Many other devices for delivery of medication to the nostril of a user are found in the prior art.

### SUMMARY OF THE INVENTION

In view of the large number of persons with medical and addiction issues, a device for naloxone/overdose reversal medication delivery where both doses are in one unit is needed. The device may be used with any medication dispensed through the nostrils of a person in need. The present arrangement provides, at least, the following advantages:
Saving on the cost of packaging as only 1 unit is needed per box as opposed to conventional devices which only include a single dose;
Saving on shipping costs;
Reduce the number of devices from going into landfills by half; and
Decrease the price over that of conventional medication delivery devices as only a single device is needed.

The device of the present arrangement allows both doses of the medication to be contained in one applicator and administered separately. This translates to saving cost, which will lower prices, increase saturation and will in turn save lives.

A device for dispensing medication is disclosed herein.

A device for dispensing medicine includes a dispensing nozzle having a hollow body and a hole at an end of the hollow body for dispensing medication. A base unit includes a base wall and a side wall. The dispensing nozzle is connected to extend from a first side of the base wall. The side wall extends around a periphery of the base wall in a direction opposite the dispensing nozzle forming a pool. The side wall includes first and second recesses on opposing sides of the side wall. A dispensing apparatus includes an actuator and a medication dispensing device extending from the actuator and into the dispensing nozzle. The actuator includes a top side having first and second arms extending outwardly therefrom. The medication dispensing device includes at least one vial including at least two doses of medication, a first needle and a second needle. The at least one vial includes first and second seals for retaining the at least two doses of medication. The actuator is movable between first and second positions. In the first position, the first and second arms of the actuator extend between the first and second recesses of the side wall and in the second position the first and second arms are rotated to extend parallel to the first and second recesses in the side wall. When in the first position, the actuator may be depressed until the first and second arms come to rest against a base of the first and second recesses, respectively, forcing the first needle to contact and pierce the first seal releasing a first dose of the at least two doses of medication through the hole in the dispensing nozzle. When the actuator is rotated into the second position, the actuator may be depressed further into the pool until the first and second arms come to rest against the base wall, forcing the second needle to contact and pierce the second seal releasing a second dose of the at least two doses of medication through the hole in the dispensing nozzle.

A device for dispensing medicine wherein the at least one vial includes a first vial and a second vial, the first seal sealing the first vial and the second seal sealing the second vial, the first vial containing the first dose of medication and the second vial containing the second dose of medication.

A device for dispensing medicine wherein the first vial and second vial are positioned back-to-back with the first seal and second seal on opposing sides thereof.

A device for dispensing medicine wherein the base side includes protrusions extending from a side thereof for gripping the device.

A device for dispensing medicine wherein the medication dispensing device includes a delivery channel positioned on a side of the first needle opposite the first seal, wherein the first and second doses of medication dispensed from the at least one vial flows through the delivery channel to the hole in the dispensing nozzle.

A device for dispensing medicine wherein the actuator includes a cylinder extending from the top side and having a ring extending around an end thereof and the first vial includes a rim extending around an end thereof, when the top side of the actuator is in the first position and depressed, the ring is moved against the rim forcing the first seal against the first needle to dispense the medication in the first vial.

A device for dispensing medicine includes a dispensing nozzle. A base unit includes a base wall and side wall. The dispensing nozzle extends from the base wall and the side wall extends from the base wall forming a pool. The side wall includes recesses on opposing sides thereof. A dispensing apparatus includes an actuator and medication dispensing device. The actuator includes arms extending outwardly therefrom. The medication retaining device retains two doses of medication. In a first position, the arms extend between the recesses. In the second position, the arms extend parallel to the recesses. In the first position, the actuator may be depressed until the arms rest against a base of the recesses dispensing a first dose of medication. In the second position, the actuator may be depressed so the arms rest against the base wall, dispensing a second dose of medication.

A device for dispensing medicine wherein the medication dispensing device includes a first vial having a first seal on one side thereof and containing a first dose of medication therein and a second vial having a second seal on one side thereof and containing a second dose of medication therein.

A device for dispensing medicine wherein the medication dispensing device includes a vial having a divider positioned therein for dividing the vial into first and second chambers, a first seal positioned on one side of the vial for retaining a first dose of medication therein and a second seal on a second side thereof, opposite the first seal for retaining a second dose of medication therein.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested elsewhere herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a device for discharging medication according to the teachings of the present disclosure.

FIG. 1 is a front top perspective view of the device for discharging medication, according to an embodiment of the disclosure.

FIG. 2 is a back bottom perspective view of the device for discharging medication, according to an embodiment of the disclosure.

FIG. 3 is a side view of the device for discharging medication, according to an embodiment of the disclosure.

FIG. 4 is a bottom view of the device for discharging medication, according to an embodiment of the present disclosure.

FIG. 5 is a top view of the device for discharging medication, according to an embodiment of the present disclosure.

FIG. 6 is a side cross-sectional view of the device for discharging medication, according to the present disclosure taken along the line SIDE-SIDE of FIG. 5.

FIG. 7 is an exploded view of the device for discharging medication, according to the present disclosure.

FIG. 8a is a top view of the device for discharging medication in a first position prior to dispensing a first dose of medication, according to the present disclosure.

FIG. 8b is a top view of the device for discharging medication in the first position after dispensing the first dose of medication, according to the present disclosure.

FIG. 8c is a top view of the device for discharging medication in a second position prior to dispensing a second dose of medication, according to the present disclosure.

FIG. 8d is a top view of the device for discharging medication in the second position after dispensing the second dose of medication, according to the present disclosure.

FIG. 9a is a top cross-sectional view of the device for discharging medication in the first position prior to dispensing a first dose of medication, according to the present disclosure.

FIG. 9b is a top cross-sectional view of the device for discharging medication in the first position after dispensing the first dose of medication, according to the present disclosure.

FIG. 9c is a top cross-sectional view of the device for discharging medication in the second position prior to dispensing a second dose of medication, according to the present disclosure.

FIG. 9d is a top cross-sectional view of the device for discharging medication in the second position after dispensing the second dose of medication, according to the present disclosure.

FIG. 10 is a flow diagram showing the method of dispensing two doses of medication according to the present disclosure.

The various embodiments of the present invention will hereinafter be described in conjunction with the appended drawings, wherein like designations denote like elements.

### DETAILED DESCRIPTION

As discussed above, embodiments of the present disclosure relate to a device for dispensing medication and more particularly to a device able to dispense at least two (2) doses of medication separately. The device of the present disclosure is designed for easily dispensing of two separate doses of medication. The device provides safeguards for dispensing a definite amount of medication with each dose. The device prevents inadvertently dispensing of a second dose after a first dose is dispensed. The device requires a definitive action after a first dose is dispensed and before a second dose can be dispensed. This advantageously enables a user to dispense two separate doses of medication such as one dose in each nostril and preventing accidental dispensing of a second dose during or after delivery of the first dose.

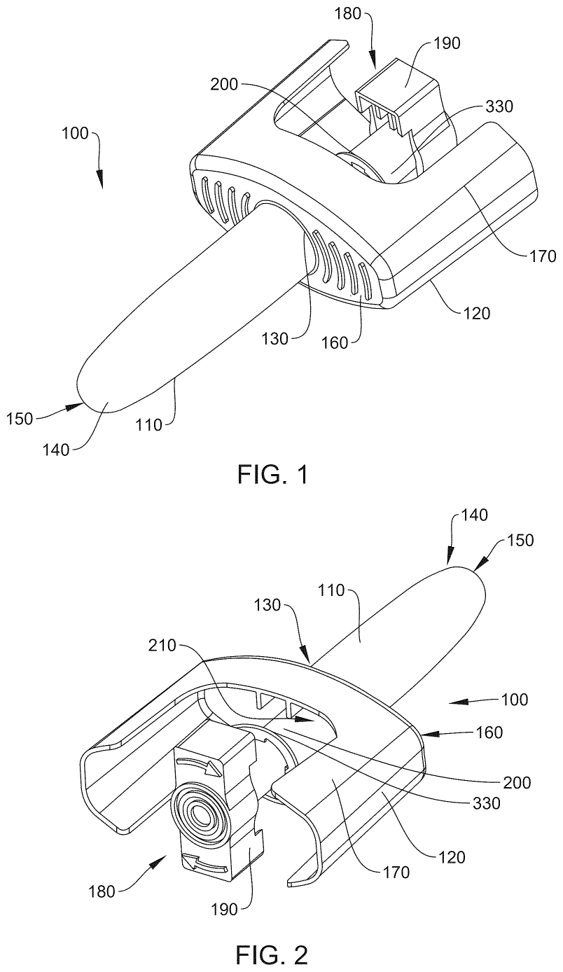

Referring now more specifically to the drawings by numerals of reference, there is shown, in FIGS. 1-10, various views of a device for dispensing medication 100.

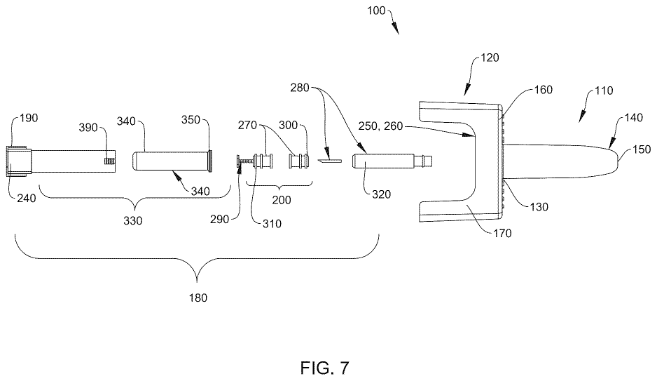

FIG. 1 shows the device for dispensing medication 100, according to an embodiment of the present disclosure. The device 100 may include a dispensing nozzle 110 extending from a base member 120. The dispensing nozzle 110 may be cylindrical in shape with an open end 130 at a connection with the base member 120 for receiving a dispensing mechanism 180 including a vial or vials containing medication. Alternatively, the dispensing nozzle 110 may extend in any geometrical shape able to receive the dispensing mechanism 180 therein. The dispensing nozzle 110 may be hollow along its length. An end 140 of the dispensing nozzle 110 opposite the connection to the base member 120 may include a recess 150 through which medication may be dispensed. The base member 120 includes a base wall 160 and a side wall 170 extending from and around a periphery of the base wall 160 in a direction opposite the connection to the dispensing nozzle 110 forming a pool within the base member 120. The dispensing mechanism 180 may be received within the pool formed by the side wall 170 of the base member 120 and extends into the dispensing nozzle 110. The dispensing mechanism 180 may include an actuator 190 for controlling dispensing of medication. The actuator 190 includes a top side 240 which may be in the form of a wing nut, plunger or any type of handle or lever able to be depressed towards the base wall 160 and into the pool of the base member 120. The actuator 190 may also include a depressor 330 extending from the top side 240 and towards the dispensing nozzle 110. A medication retaining device 200 (see FIG. 7) containing at least 2 separate doses of medication may extend into and from the depressor 330 of the actuator 190 and into the dispensing nozzle 110. The medication retaining device 200 is described as containing at least 2 separate doses of medication for purposes of example only. The medication retaining device 200 may alternatively contain at least 2 doses of any liquid or gas depending on the intended use of the device. The device may be used with any medication dispensed through the nostrils of a person in need.

FIG. 2 shows a back side perspective view of the device for dispensing medication 100, according to an embodiment of the present disclosure. The device 100 may include the dispensing nozzle 110 extending from the base member 120. The dispensing nozzle 110 may be cylindrical in shape with an open end 130 at a connection with the base member 120 for receiving the dispensing mechanism 180 including the vial or vials which may contain at least 2 doses of medication. The dispensing nozzle 110 may be hollow along its length. The end 140 of the dispensing nozzle 110 opposite the connection to the base member 120 may include the recess 150 through which medication may be dispensed. The base member 120 may include the base wall 160 and the side wall 170 extending from and around the periphery thereof in a direction opposite the connection to the dispensing nozzle 110 forming the pool. The dispensing mechanism 180 may be received within the pool formed by the side wall 170 of the base member 120 and extends into the dispensing nozzle 110. The base wall 160 may include a recess 210 aligned with the connection to the dispensing nozzle 110 through which the dispensing mechanism 180 may extend into the dispensing nozzle 110. The actuator 190 includes the top side 240 which may be in the form of a wing nut, plunger or any type of handle or lever able to be depressed towards the base wall 160 and into the pool of the base member 120. The depressor 330 extends from the top side 240 and towards the dispensing nozzle 110. The medication retaining device 200 may extend into and from the depressor 330 and into the dispensing nozzle 110.

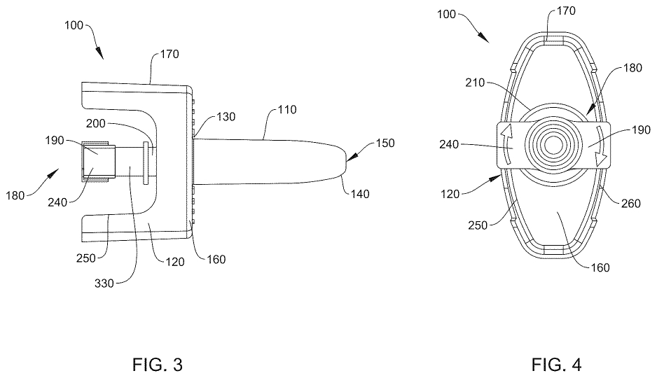

FIG. 3 shows a side view of the device for dispensing medication 100, according to an embodiment of the present disclosure. The device 100 may include the dispensing nozzle 110 extending from the base member 120. The dispensing nozzle 110 may be cylindrical in shape with the open end 130 at a connection with the base member 120 for receiving the dispensing mechanism 180 including the vial or vials containing medication. The dispensing nozzle 110 may be hollow along its length. The opposite end 140 of dispensing nozzle 110 may include the recess 150 through which medication may be dispensed. The base member 120 may include the base wall 160 and the side wall 170 extending around the periphery thereof in a direction opposite the connection to the dispensing nozzle 110 forming the pool. The dispensing mechanism 180 may be received within the pool formed by the side wall 170 of the base member 120 and extends into the dispensing nozzle 110. The base wall 160 may include the recess 210 aligned with the connection to the dispensing nozzle 110 through which the dispensing mechanism 180 may extend into the dispensing nozzle 110. The dispensing mechanism 180 may include the actuator 190 for controlling dispensing of medication and the medication retaining device 200. The medication retaining device 200 may extend at least partially into and extend out from the actuator 190. The actuator 190 may be in the form of a wing nut, plunger or any type of handle or lever able to be depressed towards the base wall 160 of the base member 120. The medication retaining device 200 may extend into and from the actuator 190 and into the dispensing nozzle 110. The actuator 190 may include the top side 240 including first and second arms extending outwardly therefrom. The depressor 330 may extend from the top side 240 with the medication retaining device 200 extending into and from the depressor 330 of the actuator 190 and into the dispensing nozzle 110. The top side 240 may be rotated between a first position in which the device is enabled for delivering a first dose of medication and a second position in which the device is enabled for dispensing a second dose of the medication. The side wall 170 may include a first recess 250 cut into a first side thereof and a second recess 260 cut into a side of the side wall 170 opposite the first recess 250. In the first position, the arms of the top side 240 may extend between the first and second recesses, 250 and 260, respectively. When the top side 240 is in the first position and is depressed to dispense the first dose of medication, a base of the first and second recesses, 250 and 260, respectively, may contact first and second arms, respectively, of the top side 240 to limit the distance the actuator 190 may extend into the dispensing nozzle 110 and thereby enable dispensing of only the first dose of medication. The top side 240 may then be rotated into the second position with the first and second arms extending parallel to the first and second recesses, 250 and 260, respectively, enabling the actuator 190 to move further into the pool. In the second position, when the actuator 190 is depressed to dispense the second dose of medication, the first and second arms of the top side 240 may be received further into the pool, causing the actuating mechanism 190 to extend further into the dispensing nozzle 110 for dispensing the second dose of medication.

FIG. 4 shows a bottom view of the device for dispensing medication 100, according to an embodiment of the present disclosure. From this view, the pool formed by the side wall 170 and base wall 160 of the base member 120 can be seen. The base member 120 may include the base wall 160 and the side wall 170 extending around a periphery thereof in a direction opposite the connection to the dispensing nozzle 110 to form the pool. The dispensing mechanism 180 may be received within the pool 180 formed by the side wall 170 of the base member 120, extending through the recess 210 in the base wall 160 and into the dispensing nozzle 110. The dispensing mechanism 180 may include the actuator 190 for controlling dispensing of medication. The actuator 190 may be in the form of a wing nut, plunger or any type of handle or lever able to be depressed towards the base wall 160 of the base member 120. The medication retaining device 200 may extend into and from the actuator 190 and into the dispensing nozzle 110. The actuator 190 may include a top side 240 extending substantially perpendicular to the medication retaining device 200. The top side 240 may be rotated between the first position for delivering a first dose of medication and the second position for dispensing a second dose of the medication. The top side 240 is shown in the first position prior to dispensing of the first dose of medication. The top side includes the arms extending therefrom which, in the first position, extend atop the recesses 250, 260 of the side wall 170.

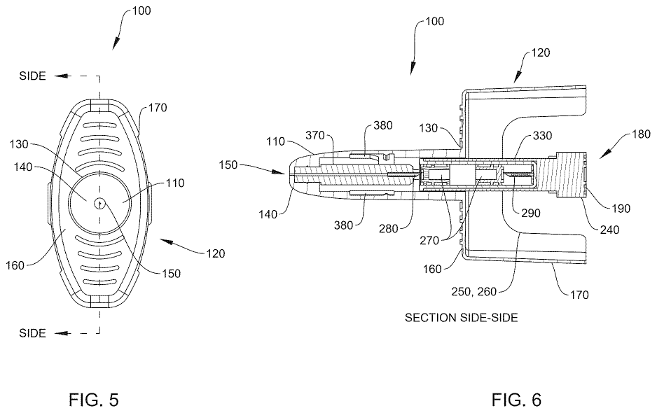

FIG. 5 shows a top view of the device for dispensing medication 100, according to an embodiment of the present disclosure. From this view, the medication dispensing end of the device can be seen. The dispensing nozzle 110 may be connected to extend from the base wall 160 of the base member 120. The dispensing nozzle 110 may be cylindrical in shape. Alternatively, the dispensing nozzle 110 may be in any geometrical shape extending from the base member 120 able to receive the dispensing mechanism 180. The dispensing nozzle 110 may include the recess 150 at the end 140 opposite the connection 130 to the base member 120 for dispensing medication released from the dispensing mechanism 180. The base member 120 includes the base wall 160 and the side wall 170 extending around a periphery thereof in a direction opposite the connection to the dispensing nozzle 110 to form the pool. A side of the base wall 160 opposite the pool may include protrusions or bumps 230 thereon for assisting a user in gripping the device 100 when dispensing medication.

FIG. 6 shows a side cross-sectional view of the device for dispensing medication 100, according to an embodiment of the present disclosure taken along the line side-side of FIG. 5. The device 100 may include the dispensing nozzle 110 extending from the base member 120. The dispensing nozzle 110 may be cylindrical in shape with the open end 130 connecting with the base member 120 for receiving the dispensing mechanism 180 including the vial or vials 270 containing medication. Alternatively, the dispensing nozzle may extend in any geometrical shape able to receive the dispensing mechanism 180 therein. The dispensing nozzle 110 may be hollow along its length. A channel 380 may extend along a portion of an inside wall of the dispensing nozzle 110. The end 140 of the dispensing nozzle 110 opposite the connection to the base member 120 may include the recess 150 through which medication may be dispensed. The base member 120 may include the base wall 160 and the side wall 170 extending from and around a periphery of the base wall 160 in a direction opposite the connection to the dispensing nozzle 110 forming the pool within the base member 120. The dispensing mechanism 180 may be received within the pool formed by the side wall 170 and base wall 160 of the base member 120 and extends into the dispensing nozzle 110. The dispensing mechanism 180 may include an actuator 190, a depressor 330, the medication retaining device 200 and a delivery channel 320 for controlling dispensing of medication. The actuator 190 may be in the form of a wing nut, plunger or any type of handle or lever able to be depressed into the pool of the base member 120 towards the base wall 160. The actuator 190 may include a top side 240. The depressor 330 may extend from the top side 240 and towards the dispensing nozzle 110. At least one nub 390 may extend from an outer wall of the depressor 330. The at least one nub 390 may be received within the channel 380 of the dispensing nozzle 110 prior to operation of the device 100 retaining the actuator 190 in the first position and preventing rotation of the actuator 190 from the first position into the second position. The medication retaining device 200 may be received at least partially within the depressor 330. The delivery channel 320 may be positioned to extend from the depressor 330 on a side of the depressor 330 opposite the top side 240. The depressor 330 and delivery channel may be received within the dispensing nozzle 110. The top side 240 of the actuator 190 may include first and second arms extending therefrom. The top side 240 may be rotated between the first position for dispensing the first dose of medication and the second position for dispensing the second dose of the medication after dispensing the first dose of medication. The side wall 170 may include the first recess 250 cut into the first side thereof and the second recess 260 cut into the side of the side wall 170 opposite the first recess 250. In the first position, the top side 240 may extend between the first and second recesses, 250 and 260, respectively with the nub 390 received within the channel 380 preventing the top side 240 from being rotated into the second position. When the top side 240 is in the first position and is depressed to dispense the first dose of medication, the actuator 190 may cause the nub 390 move along the channel 380 and the first needle 280 to engage and puncture the first seal on the medication retaining device 200 causing the first dose of medication to be dispensed therefrom, through the delivery channel 320 and out through the hole 150. Depression of the top side 240 is limited by the first and second arms coming into contact with a base of the first and second recesses, 250 and 260, respectively, to limit the distance the actuator may extend into the dispensing nozzle 110 enabling only dispensing of the first dose of medication. When the first and second arms contact the base of the first and second recesses 250 and 260, the nub 390 passes through and may be positioned below the channel 380 enabling the top side 240 to be rotated into the second position. The top side 240 may then be rotated into the second position so the arms of the top side 240 extend parallel to the first and second recesses, 250 and 260, respectively. In the second position, when the actuator 190 is depressed to dispense the second dose of medication, the first and second arms of the top side 240 may be received further into the pool and cause the second needle 290 to engage and puncture the second seal on the medication retaining device 200 causing the second dose of medication to be dispensed therefrom.

FIG. 7 shows an exploded view of the device for dispensing medication 100, according to an embodiment of the present disclosure. The device 100 may include the dispensing nozzle 110 extending from the base member 120. The dispensing nozzle 110 may be cylindrical in shape with the open end 130 at the connection with the base member 120 for receiving the dispensing mechanism 180. Alternatively, the dispensing nozzle 110 may extend in any geometrical shape able to receive at least a portion of the dispensing mechanism 180 therein. The dispensing nozzle 110 may be hollow along its length. A channel 380 (not shown in this figure) may extend along a portion of an inside wall of the dispensing nozzle 110. The end 140 of the dispensing nozzle 110 opposite the connection to the base member 120 may include the recess 150 through which medication may be dispensed. The base member 120 includes the base wall 160 and the side wall 170 extending from and around a periphery of the base wall 160 in a direction opposite the connection to the dispensing nozzle 110 forming the pool within the base member 120. The side wall 170 may include the first recess 250 cut into the first side thereof and the second recess 260 cut into the side wall 170 on a side opposite the first recess 250. The dispensing mechanism 180 may be received within the pool formed by the side wall 170 and the base wall 160 of the base member 120 and extends into the dispensing nozzle 110. The dispensing mechanism 180 may include the actuator 190, a depressor 330, the medication retaining device 200 and a delivery channel 320 for controlling dispensing of medication. The actuator 190 may be in the form of a wing nut, plunger or any type of handle or lever able to be depressed into the pool of the base member 120 towards the base wall 160. The actuator 190 may include the top side 240 including first and second arms extending outwardly therefrom. The dispensing mechanism 180 also includes the depressor 330. The depressor 330 may extend from the top side 240 and towards the dispensing nozzle 110. At least one nub 390 may extend from an outer wall of the depressor 330. The at least one nub 390 may be received within the channel 380 on the inside wall of the dispensing nozzle 110 prior to operation of the device 100 retaining the actuator 190 in the first position and preventing rotation of the actuator 190 from the first position into the second position. The depressor 330 may include a cylinder 340 receiving the medication retaining device 200 and extending from the depressor 330. The medication retaining device 200 may be received at least partially within and extend from the cylinder 340 and into the dispensing nozzle 110. The medication retaining device 200 may include at least one vile 270. If the at least one vile 270 includes one vial chamber, the one vial chamber may include a divider separating the vial chamber into a first section and a second section. The first section may include a first seal 300 on an end thereof and the second section may include a second seal 310 on an end opposite the first seal 300. Each of the first and second sections may include a dose of medication contained therein. If the at least one vial 270 includes first and second vials 270, the first and second vials may be aligned back to back. The first vial includes the first seal 300 for sealing a first dose of medication within the first vial and the second vial includes the second seal 310 for sealing a second dose of medication within the second vial. The delivery channel 320 may be positioned between the first seal 310 on the first vial and the dispensing nozzle 110. The delivery channel 320 may include a needle 280 positioned on an end adjacent the first seal 310. The cylinder 340 may include a ring 350 extending around an end thereof opposite the depressor 330. A second needle 290 may be positioned between the cylinder 340 and the second seal 310 on the second vial 270. In the first position, the top side 240 may extend between the first and second recesses, 250 and 260, respectively with the nub 390 received within the channel 380. When the top side 240 is in the first position and is depressed to dispense the first dose of medication, the actuator 190 may cause the nub 390 to move along the channel 380 and the first needle 280 to engage and puncture the first seal on the medication retaining device 200 causing a first dose of medication to be dispensed therefrom, through the delivery channel 320 and out through the hole 150. The top side 240 may be depressed until the first and second arms contact a base of the first and second recesses, 250 and 260, respectively, to limit the distance the actuator 190 may extend into the dispensing nozzle 110 enabling only dispensing of the first dose of medication. When the first and second arms contact the base of the first and second recesses 250 and 260, the nub 390 passes through and may be positioned below the channel 380 enabling the top side 240 to be rotated into the second position. The top side 240 may then be rotated into the second position with the arms of the top side 240 extending parallel to the first and second recesses, 250 and 260, respectively. In the second position, when the actuator 190 is depressed further to dispense the second dose of medication, the first and second arms of the top side 240 may be received further into the pool and cause the second needle 290 to engage and puncture the second seal on the medication retaining device 200 causing the second dose of medication to be dispensed therefrom.

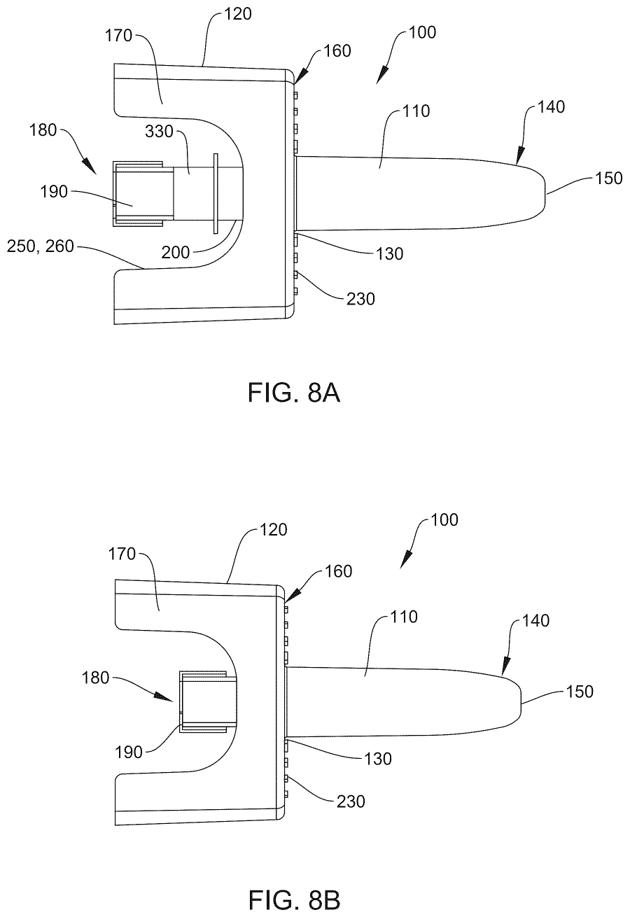

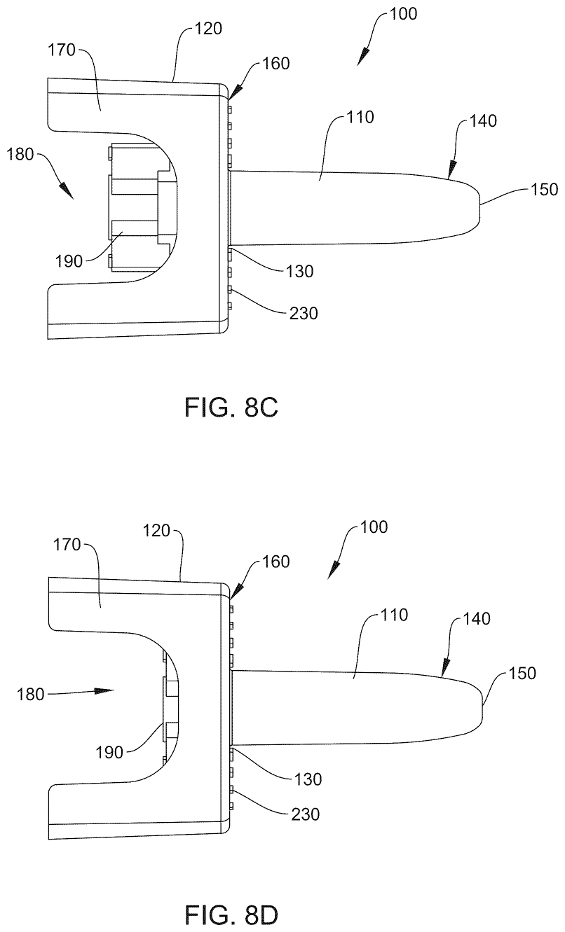

FIGS. 8a-d show side views of the device for dispensing medication 100, according to an embodiment of the present disclosure with the actuator 190 of the dispensing mechanism 180 in different positions of operation. FIG. 8a shows the actuator 190 in an initial first position prior to dispensing any medication. FIG. 8b shows the actuator in the first position after dispensing of the first dose of medication. FIG. 8c shows the actuator 190 in the second position prior to dispensing of the second dose of medication. FIG. 8d shows the actuator in the second position after dispensing of the second dose of medication.

As shown in FIG. 8a, when the actuator 190 of the device for dispensing medication 100 is in the first position, the top side 240 of the actuator 190 may be positioned near a top of the pool with the arms extending between the first and second recesses 250, 260 in the side wall 170 of the base member 120. In this position, the first and second seals 300, 310 of the at least one vial are intact with the medication contained within the at least one vial 270. The nub 390 may also be received within the channel 380 preventing the actuator 190 from being rotated into the second position.

FIG. 8b shows the device for dispensing medication 100 with the actuator 190 in the first position after dispensing of a first dose of the medication. Pressure is applied to the top side 240 of the actuator 190 towards the dispensing nozzle 110 to move the actuator into this first depressed position. Depressing the actuator 190 causes the depressor 330 including the cylinder 340 containing the at least one vial 270 to be moved towards the dispensing nozzle 110 thereby pressing the first seal of the at least one vial 270 against the first needle 280. The first needle 280 may puncture the first seal, dispensing the first dose of medication through the delivery channel 320 and the recess 150 in the dispensing nozzle 110. The actuator 190 is prevented from moving further towards the dispensing nozzle 110 when the arms of the top side 240 come to rest against the base of the first and second recesses 250, 260 of the wall 170. In this position, the arms of the top side 240 are prevented from being depressed further into the pool and the device 100 is unable to dispense the second dose of medication. When depressed into this position, the nub 390 passes through and may exit the channel 380, enabling the actuator 190 to be rotated into the second position.

In FIG. 8c the top side 240 is shown rotated into the second position with the arms extending parallel to the first and second recesses, 250 and 260, respectively. Once the top side 240 is rotated, the device 100 is enabled to dispense the second dose of medication.

FIG. 8d shows the device for dispensing medication 100 with the top side 240 in the second position and the actuator 190 further depressed into the pool. When pressure is applied to the actuator 190 with the top side 240 in the second position, the second needle 290 may be moved towards the second seal 310, causing the second needle 290 to contact and puncture the second seal 310 dispensing the medication therefrom and through the delivery channel 320 and recess 150 in the dispensing nozzle 110. The arms of the top side 240 come to rest against the base wall 160 of the base member 120.

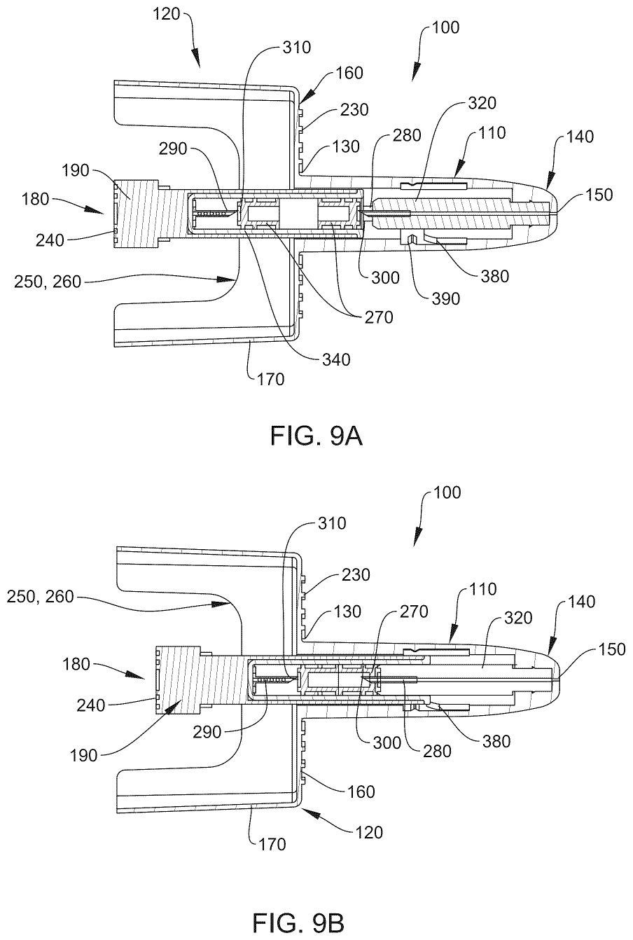

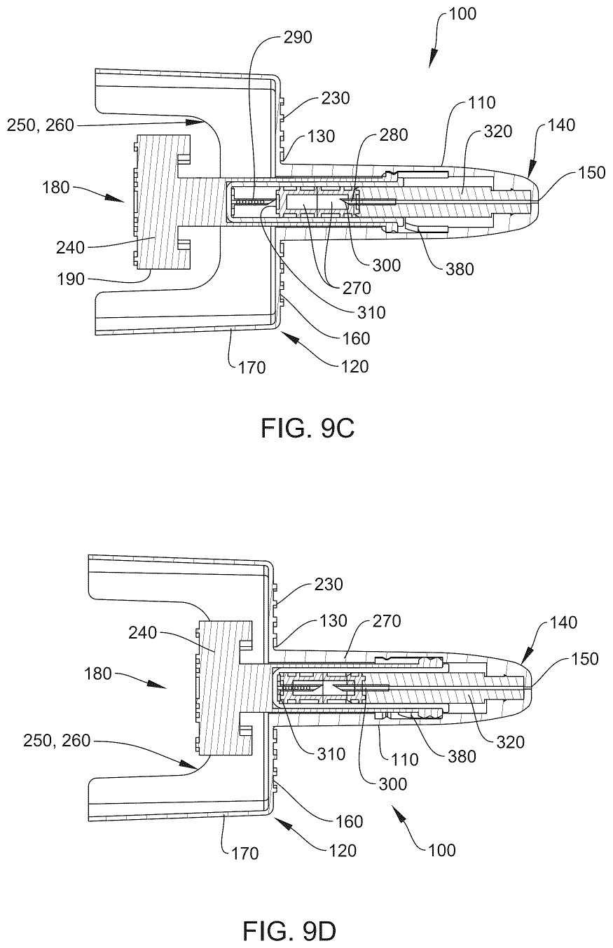

FIGS. 9a-9d show side cross sectional views of the device for dispensing medication 100 in different operating positions. FIG. 9a shows the actuator 190 in the first position prior to dispensing of medication. FIG. 9b shows the actuator 190 in the first position after dispensing of a first dose of medication. FIG. 9c shows the device with the actuator 190 in the second position, prior to dispensing a second dose of medication. FIG. 9d shows the device with the actuator 190 in the second position after dispensing of the second dose of medication.

As shown in FIG. 9a, when the actuator 190 of the device for dispensing medication 100 is in the first position, the top side 240 of the actuator 190 may be positioned near a top of the pool with the arms extending between the first and second recesses 250, 260 in the side wall 170 of the base member 120. The at least one nub 390 may be received within the channel 380 prior to operation of the device 100 retaining the actuator 190 in the first position and preventing rotation of the actuator 190 from the first position into the second position. In this position, the first and second seals 300, 310 of the at least one vial 270 are intact with the medication contained within the at least one vial 270. The first needle 280 and second needle 290 can be seen spaced from the first seal 300 and second seal 310, respectively.

FIG. 9b shows the device for dispensing medication 100 with the actuator 190 in the first position after dispensing the first dose of medication. Pressure is applied to the top side 240 of the actuator 190 towards the dispensing nozzle 110 to move the actuator into this first depressed position. Depressing the actuator 190 causes the at least one vial 270 to be moved towards the dispensing nozzle 110 thereby pressing the first seal of the at least one vial 270 against the first needle 280. The first needle 280 is shown puncturing the first seal 300, dispensing the first dose of medication through the delivery channel 320 and recess 150 in the dispensing nozzle 110. The actuator 190 is prevented from moving further towards the dispensing nozzle 110 when the arms of the top side 240 come to rest against the base of the first and second recesses 250, 260 of the wall 170. In this position, the device 100 is unable to dispense the second dose of medication. The at least one nub 390 may pass through and out of the channel 380 as the actuator 190 is depressed. Once the nub 390 passes through and out of the channel 280, the actuator may be rotated from the first position into the second position.

FIG. 9c shows the device for dispensing medication 100 with the actuator 190 in the second position prior to dispensing of the second dose. Once the top side 240 is rotated into the second position, the device 100 is enabled to dispense the second dose of medication.

FIG. 9d shows the device for dispensing medication 100 with the top side 240 in the second position and the actuator 190 further depressed into the pool. After the top side 240 is moved into the second position, when pressure is applied to the actuator 190, the second needle 290 may be moved towards the second seal 310, causing the second needle 290 to contact and puncture the second seal 310 dispensing the medication therefrom and through delivery channel 320 and the recess 150 in the dispensing nozzle 110. The arms of the top side 240 come to rest against the base wall 160 of the base member 120.

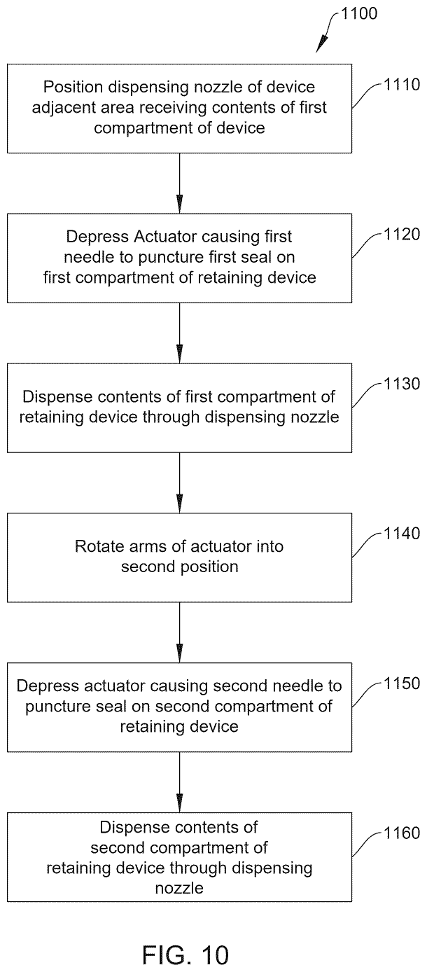

FIG. 10 is a flow chart showing the operation of the device for dispensing medication 100. With the actuator 190 in the first position with the top side 240 extending between the first and second recesses, 250 and 260, respectively, and the nub 390 received within the channel 380, the dispensing nozzle 110 is positioned adjacent the area receiving medication as shown in step 1110. When in this position, the actuator is depressed into the pool as described in step 1120. Depressing the actuator 190 causes the nub 390 to move along the channel 380 and the first needle 280 to engage and puncture the first seal on the medication retaining device 200. The first dose of medication is then dispensed therefrom, through the delivery channel 320 and out through the hole 150 as described in step 1130. The top side 240 may be depressed until the first and second arms contact a base of the first and second recesses, 250 and 260, respectively, to limit the distance the actuator 190 may extend into the dispensing nozzle 110 enabling only dispensing of the first dose of medication. When the first and second arms contact the base of the first and second recesses 250 and 260, the nub 390 passes through and may be positioned below the channel 380 enabling the top side 240 to be rotated into the second position as shown in step 1140. The top side 240 is rotated into the second position with the arms of the top side 240 extending parallel to the first and second recesses, 250 and 260, respectively. In the second position, the actuator 190 is depressed further to dispense the second dose of medication as discussed in step 1150, the first and second arms of the top side 240 may be received further into the pool and cause the second needle 290 to engage and puncture the second seal on the medication retaining device 200. The second dose of medication to be dispensed therefrom as described in step 1160.

It should be noted that the steps described in the method of use can be carried out in many different orders according to user preference. The use of “step of” should not be interpreted as “step for”, in the claims herein and is not intended to invoke the provisions of 35 U.S.C. 112(f). It should also be noted that, under appropriate circumstances, considering such issues as design preference, user preferences, marketing preferences, cost, structural requirements, available materials, technological advances, etc., other methods for using the sink are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for dispensing medicine comprising;
a dispensing nozzle including a hollow body and a hole at an end of the hollow body for dispensing medication;
a base unit including a base wall and a side wall, the dispensing nozzle connected to extend from a first side of the base wall and the side wall extending around a periphery of the base wall in a direction opposite the dispensing nozzle forming a pool, the side wall including first and second recesses on opposing sides of the side wall;
a dispensing apparatus including an actuator and a medication dispensing device extending from the actuator and into the dispensing nozzle, the actuator includes a top side having first and second arms extending outwardly therefrom, the medication retaining device includes at least one vial having at least two chambers, a first needle and a second needle, the at least one vial including first and second seals for sealing the at least two chambers, each chamber retaining a dose of medication, wherein, the actuator is movable between first and second positions, in the first position, the first and second arms of the actuator extend between the first and second recesses of the side wall and in the second position the first and second arms are rotated to extend parallel to the first and second recesses in the side wall, when in the first position, the actuator may be depressed until the first and second arms come to rest against a base of the first and second recesses, respectively, forcing the first needle to contact and pierce the first seal releasing the medication in a first chamber through the hole in the dispensing nozzle, when the actuator is rotated into the second position, the actuator may be depressed further into the pool until the first and second arms come to rest against the base wall, forcing the second needle to contact and pierce the second seal releasing the medication in a second chamber through the hole in the dispensing nozzle.

2. The device of claim 1, wherein the at least one vial includes a first vial and a second vial, the first seal sealing the first vial and the second seal sealing the second vial.

3. The device of claim 2, wherein the first vial and second vial are positioned back to back with the first seal and second seal on opposing sides thereof.

4. The device of claim 1, wherein the base side includes protrusions extending from a side thereof for gripping the device.

5. The device of claim 2, wherein the medication dispensing device includes a delivery channel positioned on a side of the first needle opposite the first seal, wherein medication dispensed from the first and second chambers flows through the delivery channel to the hole in the dispensing nozzle.

6. The device of claim 2, wherein the actuator includes a cylinder extending from the top side and having a ring extending around an end thereof and the first vial includes a rim extending around an end thereof, when the top side of the actuator is in the first position and depresses, the ring is moved against the rim forcing the first seal against the first needle to dispense the medication in the first vial.

7. The device of claim 1, wherein the dispensing nozzle further includes a channel along at least a portion of an inner wall thereof and the dispensing apparatus further includes a nub extending from a side thereof, wherein the nub is received within the channel when the actuator is in the first position preventing the actuator from rotating into the second position.

8. A device for dispensing medicine comprising;
a dispensing nozzle including a hollow body and a hole at an end of the hollow body for dispensing medication;
a base unit including a base wall and a side wall, the dispensing nozzle connected to extend from a first side of the base wall and the side wall extending around a periphery of the base wall in a direction opposite the dispensing nozzle forming a pool, the side wall including first and second recesses on opposing sides of the side wall;
a dispensing apparatus including an actuator and a medication dispensing device extending from the actuator and into the dispensing nozzle, the actuator includes a top side having first and second arms extending outwardly therefrom, the medication retaining device retaining at least two doses of medication, wherein, the actuator is movable between first and second positions, in the first position, the first and second arms of the actuator extend between the first and second recesses of the side wall and in the second position the first and second arms are rotated to extend parallel to the first and second recesses in the side wall, when in the first position, the actuator may be depressed until the first and second arms come to rest against a base dispensing a first dose of medication through the hole in the dispensing nozzle, when the actuator is rotated into the second position, the actuator may be depressed further into the pool until the first and second arms come to rest against the base wall, dispensing a second dose of medication through the hole in the dispensing nozzle.

9. The device of claim 8, wherein the medication dispensing device includes a first vial having a first seal on one side thereof and containing a first dose of medication therein and a second vial having a second seal on one side thereof and containing a second dose of medication therein.

10. The device of claim 8, wherein the medication dispensing device includes a vial having a divider positioned therein for dividing the vial into first and second chambers, a first seal positioned on one side of the vial for retaining a first dose of medication therein and a second seal on a second side thereof, opposite the first seal for retaining a second dose of medication therein.

11. The device of claim 8, wherein the dispensing nozzle further includes a channel along at least a portion of an inner wall thereof and the dispensing apparatus further includes a nub extending from a side thereof, wherein the nub is received within the channel when the actuator is in the first position preventing the actuator from rotating into the second position.

12. A method of dispensing medication comprising the activities of:
placing dispensing nozzle of delivery device adjacent a first area receiving contents of the delivery device when an actuator is in a first position;
depressing the actuator towards the dispensing nozzle until the actuator rests against a wall of a base unit causing a first needle to puncture seal on a first vial;
dispensing contents within a first vial through the dispensing nozzle;
rotating the actuator into a second position extending within the wall of the base unit;
placing the dispensing nozzle adjacent a second area receiving the contents of the delivery device;
depressing the actuator towards the dispensing nozzle causing a second needle to puncture seal on a second vial; and
dispense contents within the second vial through the dispensing nozzle.

13. The method as claimed in claim 12, wherein when said actuator is in said first position and prior to said actuator being depressed, a nub on said actuator is retained within a channel on an inner side of said dispensing nozzle preventing said actuator from rotating into said second position and dispensing said second dose of the contents of the delivery device.

14. The method as claimed in claim 13, wherein, when said actuator is in said first position and depressed, said nub is caused to move through and exit said channel when said actuator rests against the wall of the base unit enabling said actuator to be rotated into said second position.

15. The method of claim 13, wherein the contents of the delivery device is a medication.
